(19) United States
(12) Patent Application Publication
(10) Pub. No.: US 2021/0371853 A1
(43) Pub. Date: Dec. 2, 2021

(54) METHODS FOR NUCLEIC ACID LIBRARY CREATION

(71) Applicant: Viome, Inc., Bellevue, WA (US)

(72) Inventors: Brittany TWIBELL, Santa Fe, NM (US); Ryan TOMA, Los Alamos, NM (US); James HORNE, Los Alamos, NM (US); Andrew HATCH, Santa Fe, NM (US); Momchilo VUYISICH, Bothell, WA (US)

(21) Appl. No.: 15/733,182

(22) PCT Filed: Dec. 8, 2018
(86) PCT No.: PCT/US2018/064638

§ 371 Date: Jun. 4, 2020

Related U.S. Application Data
(60) Provisional application No. 62/596,795, filed on Dec. 9, 2017.

(51) Int. Cl. C12N 15/10; C12Q 1/6806

(52) U.S. Cl. CPC C12N 15/1096; C12Q 1/6806

(57) ABSTRACT

Provided herein are methods of making nucleic acid libraries. Libraries can be enriched for target nucleic acids by subtractive purification involving the use of poly-tagged capture probes and/or a DNA degradation step performed after a subtractive purification step.

### REFERENCE TO RELATED APPLICATIONS

This application claims the benefit of the priority date of U.S. provisional patent application 62/596,795, filed Dec. 9, 2017.

### STATEMENT AS TO FEDERALLY SPONSORED RESEARCH

None.

### BACKGROUND

High throughput DNA sequencing has made it possible to analyze nucleic acid samples from subjects for diagnostic, wellness and recreational purposes. Subject samples containing nucleic acids need to be preserved to prevent nucleic acid degradation before laboratory analysis. Preserving nucleic acids is particularly important for companies offering consumers tests in which samples collected at home are transmitted to a laboratory for analysis. The time between sample collection and analysis can be days or weeks. Accordingly, kits provided to individuals preferably include simple methods for individuals to preserve their nucleic acids.

Preparation of nucleic acid libraries can involve a negative or positive selection step in which undesired species are captured and removed from a sample or desired species are captured and isolated. Negative selection methods can involve the use of nucleic acid probes tagged with extraction moieties. For a variety of reasons nucleic acid probes can be left behind in the sample with a desired species. Such contaminated molecules can find their way into nucleic acid libraries where they constitute irrelevant or misleading information.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary protocol for creating and analyzing a library of RNA sequences. I'm cozy

FIG. 2 shows an exemplary protocol for preparing and using poly-tagged probe ensembles.

FIG. 3 shows an oligonucleotide probe comprising four extraction moieties (biotin (“B”) attached substantially evenly across the molecule at the ends of the polynucleotide and internally at the middle third portion of the polynucleotide.

FIG. 4 shows an exemplary protocol for using poly-tagged probe ensembles.

### SUMMARY

In one aspect, provided herein is a method of preparing a cDNA library comprising: (a) providing a sample containing RNA; (b) optionally, disrupting cells in the sample; (d) degrading initial DNA in the isolated polynucleotides to produce an RNA-enriched sample; (e) contacting the RNA-enriched sample with an ensemble of oligonucleotide probes, wherein the oligonucleotide probes hybridize with and capture non-target RNA species in the sample and wherein the ensemble comprises oligonucleotide probes bearing two or more extraction moieties; (f) removing captured non-target RNA species using the extraction moiety, thereby producing a target RNA-enriched sample; (g) optionally, degrading remaining DNA in the target RNA-enriched sample; (h) converting RNA in the target RNA-enriched sample into cDNA molecules; and (i) attaching adapters to the cDNA molecules to produce adapter-tagged cDNA molecules, thereby producing cDNA library. In one embodiment the sample comprises RNA from a subject (e.g., human or animal). In another embodiment the subject is a human or nonhuman mammal. In another embodiment the subject is a host, and the sample comprises both host RNA and microbial RNA. In another embodiment the sample comprises a cultured biological material, an environmental sample, an agricultural sample or a forensic sample. In another embodiment the sample comprises capillary blood, venous blood or arterial blood. In another embodiment the sample comprises from about 1 μL to about 100 μL (e.g., about 5 μL to about 75 μL or about 20 μL to about 50 μL) of blood. In another embodiment the sample further comprises an RNA preservative. In another embodiment the RNA preservative comprises formalin, sulfate (e.g., ammonium sulfate) or isothiocyanate (e.g., guanidinium isothiocyanate). In another embodiment providing the sample comprises performing a skin prick and collecting the blood into a capillary tube. In another embodiment the method further comprises sending the capillary tube via a common carrier to a central collection location. In another embodiment the method comprises disrupting cells, e.g., by performing bead beating (e.g., with zirconium beads) or ultrasonic lysis. In another embodiment the method comprises degrading initial DNA and/or remaining DNA, e.g., by treatment with a DNase (e.g., DNase I (Sigma-Aldrich), Turbo DNA-free (ThermoFisher) or RNase-Free DNase (Qiagen)). In another embodiment isolating polynucleotides comprises contacting the sample with magnetic particles (e.g., silica beads) that have nucleic acid binding affinity for bind the polynucleotides, and separating bound polynucleotides from unbound material. In another embodiment at least 90% (e.g., at least 95%, at least 96%, at least 97%, at least 98%, or at least 99%) of the oligonucleotide probes in the ensemble bear at least one extraction moiety. In another embodiment at least 50%, at least 60% or at least 75% of the oligonucleotide probes in the ensemble bear more than one extraction moiety. In another embodiment the extraction moiety is selected from the group consisting of biotin, streptavidin, avidin, a magnetically attractable particle, a peptide, and an antibody. In another embodiment non-target RNA species include one or more of: human ribosomal RNA (rRNA), human transfer RNA (tRNA), microbial rRNA, and microbial tRNA. In another embodiment non-target RNA species further include one or more of the most abundant mRNA species in the sample. In another embodiment the most abundant mRNA species removed comprise hemoglobin and/or myoglobin. In another embodiment the most abundant mRNA species removed comprise one or more of (e.g., at least 3 of, at least 4 of, at least 5 of, at least 6 of, or all of) HFM1, PDE3A, HBB, MALAT1, ATP8/ATP6, ND4L and COX1. In another embodiment captured polynucleotides represent at least 90% of polynucleotide molecules in the RNA-enriched sample. In another embodiment adapters comprise sample barcode sequences so that each adapter-tagged cDNA molecule comprises a sample barcode. In another embodiment adapters comprise sequencing platform-specific sequences necessary and/or sufficient for sequencing on a sequencing platform. In another embodiment sequencing platform-specific sequences comprise one or more of a sequencing primer hybridization site and a cluster primer binding site. In another embodiment attaching adapters comprises performing primer extension on RNA molecules using primers comprising adapter sequences or ligating adapters to double stranded cDNA molecules. In another embodiment the method further comprises (j) sequencing the cDNA library. In another embodiment the method comprises sequencing the cDNA library to a re-depth of at least 10 million reads per sample. In another embodiment the method comprises pooling a plurality of different cDNA libraries, each library comprising a different sample barcode and sequencing the pooled cDNA libraries simultaneously. In certain embodiments, the most abundant RNA species that account for at least 90% of total RNA are removed, such that the enriched sample comprises less abundant species accounting for the bottom 10% of total RNA based on rank order. In a blood sample, for example, these lower rank abundant species can include between about 1000 to 4000 different mRNA species.

In another aspect provided herein is a cDNA library comprising adaptor-tagged DNA molecules, wherein the DNA molecules comprise nucleotide sequences of RNA molecules from animal, e.g., mammalian, e.g., human blood, and wherein fewer than any of 50%, 40%, 30%, 20%, 10%, 5%, 4%, 2% or 1% of the sequences in the library are represented by one or more (e.g., at least three, at least four, or all of) nucleotide sequences of RNA selected from the group consisting of host rRNA, microbial rRNA, host tRNA, microbial tRNA and one or more most abundant host mRNA species. In another embodiment the cDNA library further comprises trace amounts (e.g., detectable but less than 1%) of DNA probes, each probe comprising one or a plurality of extraction moieties. In another embodiment, compared with an initial RNA library from which it was derived, the cDNA library has fewer than any of 80%, 90%, 95%, 90%, or 99% of the original species of host rRNA, microbial rRNA, host tRNA, microbial tRNA or any of the 10, 15, 20 or 25 most abundant host mRNA species.

In another aspect provided herein is a method of preparing a cDNA library comprising: a) providing a sample containing DNA and RNA; b) degrading DNA in the sample to produce an RNA-enriched sample; c) contacting the RNA-enriched sample with oligonucleotide probes, wherein the oligonucleotide probes hybridize with and capture non-target RNA species in the sample; d) removing captured RNA species to produce a target RNA-enriched sample; e) degrading DNA remaining in the target RNA-enriched sample; f) converting RNA in the target RNA-enriched sample into cDNA molecules; and g) attaching adapters to the cDNA molecules, thereby producing a cDNA library.

In another aspect provided herein is a method of negative selection comprising: (a) contacting a sample with an ensemble of capture probes, wherein: (i) the capture probes selectively bind non-target molecules in the sample compared with target molecules in the sample; and (ii) a majority of the capture probes in the ensemble bear a plurality of extraction moieties and a minority of the capture probes in the ensemble bear one or no extraction moieties; and (b) separating bound non-target molecules from unbound target molecules by extracting capture probes with bound non-target molecules using the extraction moiety, to produce a target-enriched sample. In one embodiment a plurality of the capture probes comprise at least three, at least four or at least five extraction moieties. In another embodiment one or a plurality of the labels is an internal label not attached to a terminal nucleotide of the polynucleotide. In another embodiment the probes comprise oligonucleotide probes and the internal label is attached within the central 50%, central 40%, central 20% of the polynucleotide, or within two nucleotides of the nucleotide positioned at the median of the polynucleotide. In another embodiment the method further comprises: (c) removing un-extracted capture probes from the enriched sample. In another embodiment removing comprises degrading the un-extracted probes. In another embodiment degrading comprises degrading DNA with a DNase. In another embodiment the target molecules comprise microbial mRNA, the non-target molecules comprise RNA species selected from rRNA, tRNA and most abundant host mRNA species. In another embodiment the extraction moiety is selected from biotin, streptavidin, a magnetically attractable particle, a peptide, and an antibody.

In another aspect provided herein is an ensemble of polynucleotide probes wherein at least 90% (e.g., at least 95%, at least 96%, at least 97%, at least 98%, or at least 99%) of the probes bear at least one extraction moiety. In one embodiment a majority of the probes bear at least two extraction moieties and a minority of the probes bear fewer than two extraction moieties (e.g., fewer than 50%, 40%, 30%, 20%, 10%, or 5% bear one extraction moiety and/or fewer than any of 6%, 5%, 4%, 3%, 2%, or 1% bear no extraction moiety). In another embodiment at least 50%, at least 60%, at least 75%, at least 80%, at least 90%, or at least 95% of the probes in the ensemble bear at least two extraction moieties. In another embodiment the polynucleotide probes comprise sequences that hybridize and bind to non-target RNA sequences.

In another aspect provided herein is a polynucleotide probe comprising a polynucleotide and a plurality of labels attached thereto, wherein one or a plurality of the labels is an internal label not attached to a terminal nucleotide of the polynucleotide. In one embodiment the internal label is attached within the central 50%, central 40%, central 20% of the polynucleotide, or within two nucleotides of the nucleotide positioned at the median of the polynucleotide. In another embodiment the labels are distributed substantially evenly across the probe.

In another aspect provided herein is a method of generating a poly-tagged probe comprising: (a) providing an initial nucleotide or an oligonucleotide chain (collectively, “growing oligonucleotide”), wherein the growing oligonucleotide optionally comprises at least one nucleotide comprising a label; (b) iteratively coupling to the growing oligonucleotide a nucleotide, wherein at one or a plurality of coupling iterations, the nucleotide coupled comprises a label, wherein a poly-tagged probe is produced. In one embodiment the nucleotide is a deoxyribonucleotide or a ribonucleotide. In another embodiment the method comprises at least 3, at least 4, at least 5, at least 6 coupling steps comprising a labeled nucleotide. In another embodiment the label comprises an extraction moiety, e.g., biotin. In another embodiment the poly-tagged probes comprise at least 3, at least 4 or at least 5 labels. In another embodiment labeled nucleotides are coupled substantially evenly across the probe. In another embodiment the labeled nucleotides are coupled in a middle portion of the probe. In another embodiment, the method is performed on an ensemble of growing oligonucleotides, the ensemble comprises at least 100, at least 1000, at least 10,000, at least 100,000, or at least 1 million growing oligonucleotides. In another embodiment, after a plurality of iterative couplings (e.g., after assembly of the probes is complete) the ensemble comprises a plurality of oligonucleotides each of which comprises a plurality of labels, and a plurality of oligonucleotides, each of which comprises no more than one label, and wherein a majority of the oligonucleotides (e.g., at least 50% at least 60% at least 70% at least 80% at least 90% at least 95%) comprise a plurality of labels and a minority of the oligonucleotides (e.g., fewer than 50%, fewer than 40%, fewer than 30%, fewer than 20% fewer than 10% or fewer than 5%) of the oligonucleotides comprise no more than one label.

In another aspect provided herein is a method comprising: (a) providing a sample comprising nucleic acid; (b) contacting the sample with an ensemble of poly-tagged oligonucleotide probes; wherein the probes capture non-target nucleic acid molecule species in the sample; (c) separating captured non-target nucleic acid species from target nucleic acid species. In one embodiment the probes comprise RNA oligonucleotides.

In another aspect provided herein is a kit comprising: a) a lancet; b) a container containing an RNA preservative; and c) a mailing container. In one embodiment the kit further comprises b) an EDTA-coated capillary tube. In another embodiment the capillary tube comprises a Minivette™ point-of-care tool. In another embodiment the kit further comprises disinfectant wipes.

In another aspect provided herein is a method comprising: (a) providing a sample comprising polynucleotides (RNA molecules or cDNA molecules) wherein the most common polynucleotide species to the least common polynucleotide species span a dynamic range of at least any of 10³, 10⁴, 10⁵, 10⁶ or 10⁷; (b) removing from the sample most common polynucleotide species accounting for at least 90% of the total abundance of polynucleotides to produce a sample comprising uncommon polynucleotide species; and (c) sequencing the uncommon polynucleotide species. In one embodiment, removing comprises removing species accounting for at least 99% of the total abundance. In another embodiment the low abundance polynucleotide species comprise sequences for between about 1000 and about 5000 different genes. In another embodiment removing the most common polynucleotide species does not comprise positively selecting uncommon polynucleotide species. In another embodiment the uncommon polynucleotide species comprise species within the lowest 10%, 5% or lowest 1% of abundance.

### DETAILED DESCRIPTION

### I. Definitions

As used herein, the term “sample” refers to a composition comprising an analyte. A sample can be a raw sample, in which the analyte is mixed with other materials in its native form (e.g., a source material), a fractionated sample, in which an analyte is at least partially enriched, or a purified sample in which the analyte is at least substantially pure.

Samples used as source material include, without limitation, biological materials from an organism, cultured biological materials (e.g., cultured cells), environmental samples (e.g., water, soil or air), agricultural samples (e.g., a sample taken from a farm) or forensic samples (e.g., blood, hair, semen). A biological sample from an organism can comprise, for example, stool, blood, serum, plasma, saliva, throat swab, nasopharyngeal swab, sputum, pleural effusion, bronchial lavage or aspirates, urine, feces, breast milk, colostrum, tears, peritoneal fluid, cerebrospinal fluid, seminal fluid, amniotic fluid, vaginal samples, nail clippings, hoof swabs, skin or skin scrapings and/or a biopsy (e.g., tissue biopsy or liquid biopsy). In embodiments in which analysis of a subject individual's microbiome is desired, the sample can be one known to contain microorganisms, e.g., a blood microbiome or a gut microbiome.

As used herein, the term “blood” refers to whole blood or a fraction thereof, such as serum or plasma. The term “capillary blood” refers to blood taken from a capillary. The term “venous blood” refers to blood taken from a vein. The term “arterial blood” refers to blood taken from an artery.

As used herein, the term “subject” refers to an individual organism, e.g., an animal, a plant or a microbe. Animal subjects include, without limitation, human and nonhuman animals. Nonhuman animals may be non-human mammals, birds, fish, reptiles and insects. Nonhuman animals include, for example, bovines, swine, horses, sheep, goats, chickens, turkeys, dogs, cats and birds.

As used herein, the term “host” refers to an organism hosting a microbial community.

As used herein, the term “microbiome” refers to a microbial community comprising one or a plurality of different microbial strains or species inhabiting a host.

As used herein, the terms “polynucleotide” and “nucleic acid” are used interchangeably and refer to both single-stranded and double-stranded molecules. As used herein, the term “oligonucleotide” refers to short polynucleotides, e.g., no more than 500 nucleotides in length. In certain embodiments, a polynucleotide can comprise natural or non-natural nucleotides, such as peptide nucleic acids or locked nucleic acids.

As used herein, a chemical entity, such as a polynucleotide or polypeptide, is “substantially pure” if it is the predominant chemical entity of its kind in a composition. For example, a polynucleotide can be the predominant biomolecule in a composition, or RNA can be the predominant nucleic acid in a composition, or polynucleotides with particular sequences can be the predominant nucleotide sequences in a composition. This includes the chemical entity representing more than 50%, more than 80%, more than 90% or more than 95% or of the chemical entities of its kind in the composition. A chemical entity is “essentially pure” if it represents more than 98%, more than 99%, more than 99.5%, more than 99.9%, or more than 99.99% of the chemical entities of its kind in the composition. Chemical entities which are essentially pure are also substantially pure.

As used herein, the term “cDNA” refers to DNA, at least one strand of which has a nucleotide sequence copy of an RNA molecule.

As used herein, “cell-free nucleic acid” (e.g., “cell-free DNA” (“cfDNA”) or “cell-free RNA”) refers to nucleic acid not encapsulated in a cell and found in a bodily fluid, e.g., blood or urine. Cell-free DNA comprises DNA having a size range between about 120 and about 180 nucleotides.

As used herein, the term “RNA preservative” refers to a compound or composition that inhibits degradation of RNA. RNA preservatives include, without limitation, formalin, sulfate (e.g., ammonium sulfate), isothiocyanate (e.g., guanidinium isothiocyanate) and urea. Commercially available RNA preservatives include, for example, TRIzol (ThermoFisher), RNAlater (Ambion, Austin, Tex., USA), Allprotect tissue reagent (Qiagen), PAXgene Blood RNA System (PreAnalytiX GmbH, Hombrechtikon), and RNA/DNA Shield® (Zymo Research, Irvine, Calif.).

As used herein, the term “probe” refers to a nucleic acid molecule bearing a label. Typically, a probe comprises a nucleotide sequence that hybridizes to a nucleic acid molecule to be captured.

As used herein, the term “label” refers to a chemical moiety attached to a molecule, such as a nucleic acid molecule. In some embodiments, most molecular species in an ensemble bear the same label.

As used herein, the term “extraction moiety” refers to a label that can be captured or immobilized. Extraction moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. Magnetically attractable particles can be immobilized by applying magnetic force. Large particles can be captured, for example, by centrifugation. In certain embodiments, extraction moieties function as indirect detectable labels.

As used herein, the term “detectable label” refers to a label detectable by spectroscopic, photochemical, biochemical, immunochemical, chemical, or other physical means. Examples of detectable labels include, without limitation, colorimetric, fluorescent, chemiluminescent, enzymatic, and radioactive labels. A detectable label can produce a signal directly (a “direct label”) or indirectly (an “indirect label”). A direct label directly produces a signal. Examples of direct labels are fluorescent labels (e.g., phycoerythrin, fluorescein isothiocyanate, texas red, rhodamine, a green fluorescent protein, a red fluorescent protein, a yellow fluorescent protein), luminescent labels (e.g., luminescent proteins such as luciferase), enzymatic labels (e.g., horse radish peroxidase or alkaline phosphatase), colorimetric labels such as colloidal gold or colored glass or plastic (e.g., polystyrene, polypropylene, latex, etc.) beads and radioactive labels (e.g., ³H, ¹²⁵I, ³⁵S, ¹⁴C, or ³²P). In one embodiment, the detectable label is a molecular beacon comprising a nucleotide hairpin structure having tethered to its ends a fluorophore and a quencher. An indirect label is a label that is detected (primarily or secondarily) by another moiety comprising a direct label. Examples of indirect labels are extraction moieties, such as antibodies, biotin or streptavidin, that bind other molecules which themselves bear a direct label.

Detectable labels can be measured as follows. Fluorescence: A fluorescent molecule (fluorophore), such as a dye or a protein, is excited with light of specific wavelength. The fluorophore then emits light of a specific wavelength, which can be measured using a detector, such as a photomultiplier tube, CMOS, etc. Luminescence: Chemical reactions can produce light. One example is the enzyme Luciferase, that oxidizes luciferin and emits photons. This light can be measured using a detector, such as a photomultiplier tube, CMOS, etc.

As used herein, the term “ensemble” refers to a collection of individual items, e.g., molecules, which may be the same or different. For example, an ensemble of polynucleotide probe molecules refers a collection of individual probe molecules that may have the same nucleotide sequences or different nucleotide sequences. As used herein, the term “probe ensemble” includes ensembles of oligonucleotides comprising probes and in which a portion of the oligonucleotides do not comprise a label.

As used herein, the term “poly-tagged probe” refers to a probe bearing a plurality of labels (e.g., extraction moieties). As used herein, the term “poly-tagged probe ensemble” refers to an ensemble of probe molecules in which a majority of probes bear two or more (e.g., at least 2, at least 3, at least 4 or at least 5) labels, e.g., extraction moieties. In certain embodiments, a minority of the probes in the ensemble bear one or no labels, e.g., no extraction moieties.

As used herein, the term “non-informative RNA” refers to a form of non-target or non-analyte species of RNA. Non-informative RNA species can include one or more of: human ribosomal RNA (rRNA), human transfer RNA (tRNA), microbial rRNA, and microbial tRNA. Non-informative RNA species can further comprise one or more of the most abundant mRNA species in a sample, for example, hemoglobin and myoglobin in a blood sample.

As used herein, the terms “most abundant species” or “most abundant genera” refers to any one or more species or genera of molecules in a sample among those ranked from most abundant to least abundant, and that account for at least any of 50%, 75%, or at least 90% of the species.

As used herein, the term “nucleic acid library” refers to a collection of adapter-tagged polynucleotides. Typically, polynucleotide members of a nucleic acid library comprise a sample index. Optionally, they may comprise molecular barcodes useful for distinguishing individual molecules from each other, either using the barcode, alone, or in combination with sequence information from a polynucleotide insert.

As used herein, the term “adapter-tagged polynucleotide” refers to a polynucleotide comprising a nucleic acid insert flanked on one or both ends by adapter sequences bearing a primer binding site.

As used herein, the term “adapter” refers to a polynucleotide comprising adapter sequences comprising, at least, a primer binding site, e.g., a universal primer binding site or a forward or reverse primer binding site. Adapters also can comprise other elements including, without limitation, a sample barcode, a molecular barcode, a sequencing primer binding site (which may also serve as an amplification primer binding site) or a binding site for binding polynucleotide to platform hardware, such as a flow cell probe binding site. In certain embodiments, adapters can comprise non-complementary ends. These include, for example, “Y-shaped” adapters or adapters which fold back upon themselves to form looped structures. Y-shaped adapters, in particular, can be useful when different strands (“Watson” and “Crick” strands) of a double stranded nucleic acid need to be distinguished. Depending on context, the term “adapter” may also refer to a nucleotide sequence comprising adapter elements.

As used herein, the term “primer binding site” refers to a nucleotide sequence to which a polynucleotide primer can hybridize, e.g., for PCR or primer extension.

As used herein, the term “primer” refers to a polynucleotide, typically an oligonucleotide, having a sequence (“binding sequence”) that binds to a primer binding site. Primers are typically categorized as “universal primers” or “degenerate primers”. Primers are used for primer extension and PCR. In amplification, such as PCR, primers bind to primer binding sites on each strand of a double stranded nucleic acid molecule with a target sequence (amplicon) positioned between them. In certain embodiments, for example, when the primer binding site on the first strand of a double stranded molecule is different than the primer binding site on a second, complementary, strand, primers are provided as a set of two primers (“primer pair”). Primers in the primer pair may be differentiated as a “forward primer” and a “reverse primer”.

As used herein, the term “universal primer” refers to a primer having a binding sequence that binds to a primer binding site on an adapter. Accordingly, a universal primer can be used to amplify all adapter-tagged polynucleotides in a sample.

As used herein, the term “degenerate primer” refers to a mixture of primers having a substitution of different nucleotides at the binding sequence. For example, degenerate primers can have a degenerate hexamer nucleotide sequence.

As used herein, the term “barcode” refers to a nucleotide sequence which provides information about the polynucleotide in which the barcode is incorporated. A barcode may provide information specific to a single molecule or collection of molecules. Barcodes are typically provided in polynucleotide adapters. Barcodes typically have sequences of no more than 100, 50, 20 or 10 nucleotides.

As used herein, the term “sample barcode” refers to a barcode that distinguishes polynucleotides sourced from a first sample from polynucleotides sourced from a second, different sample. Accordingly, sample barcodes in an ensemble of adapters will be the same in each sample and different between different samples. For example, polynucleotides sourced from each of 50 different samples may comprise 50 different sample barcodes.

As used herein, the term “molecular barcode” refers to a barcode that, alone or in combination with other information, distinguishes different molecules in a sample from each other. For example, a set of molecular barcodes may have sufficient diversity such that substantially all molecules in a sample bear a different molecular barcode. A collection of such polynucleotides is referred to as being “uniquely tagged”. Alternatively, a set of barcodes may have a diversity that is less than the number of polynucleotides in a sample. In this case, different molecules that bear the same molecular tag may be distinguished based on information derived from the sequence of the insert. A collection of such polynucleotides is referred to herein as being “non-uniquely tagged”.

As used herein, the term “index” refers to one or more pieces of information, such as barcodes, which, alone or in combination, provide information. For example, an adapter-tagged polynucleotide can comprise a single sample barcode and/or molecular barcode, or a plurality of sample barcodes or molecular barcodes, e.g., attached at each end. A single barcode or a combination of barcodes attached to a molecule can function as an “index”. Thus, a “sample index” can be defined by one or a plurality (e.g., two) of sample barcodes, and a “molecular index” can be defined by one or a plurality (e.g., two) of molecular barcodes.

As used herein, the term “high throughput sequencing” refers to the simultaneous or near simultaneous sequencing of thousands of nucleic acid molecules. High throughput sequencing is sometimes referred to as “next generation sequencing” or “massively parallel sequencing”. Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing (PacBio), and nanopore DNA sequencing (e.g., Oxford Nanopore).

As used herein, the term “kit” refers to a collection of items intended for use together. The items in the kit may or may not be in operative connection with each other. A kit can comprise, e.g., reagents, buffers, enzymes, antibodies, probes and other compositions specific for the purpose. A kit can also include instructions for use and software for data analysis and interpretation. A kit can further comprise samples that serve as normative standards. Typically, items in a kit are contained in primary containers, such as vials, tubes, bottles, boxes or bags. Separate items can be contained in their own, separate containers or in the same container. Items in a kit, or primary containers of a kit, can be assembled into a secondary container, for example a box or a bag, optionally adapted for commercial sale, e.g., for shelving, or for transport by a common carrier, such as mail or delivery service.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”.

### II. Methods of Preparing RNA Libraries

Provided herein are methods of preparing libraries comprising sequences of RNA molecules. The RNA molecules whose sequences comprise the library can be from any source and can include all RNA from the source sample or a subset of RNA molecules from the source sample. In certain embodiments the library comprises transcriptome sequences, more particularly, sequences enriched for mRNA molecules.

Preparation of an RNA library can involve: (1) removing DNA from a sample comprising RNA and DNA to produce an RNA-enriched sample; (2) removing non-target (e.g., non-informative) RNA from the RNA-enriched sample using either singly-tagged or poly-tagged probe ensembles to produce an RNA-target enriched sample. In a first variation of the method, if singly tagged probe ensembles are used, then the method further comprises (3) performing a second DNA removal step. In a second variation of the method, a poly-tagged probe ensemble is used and the method further comprises (3) performing a second DNA removal step. This variation is particularly useful when a probe ensemble is used in which a majority of probes bear exactly two extraction moieties. In a third variation of the method, a poly-tagged probe ensemble is used and no second DNA removal step is performed. This variation is particularly useful when the probe ensemble used has a majority of probes bearing at least three extraction moieties, one of which is attached at a middle portion of the polynucleotide.

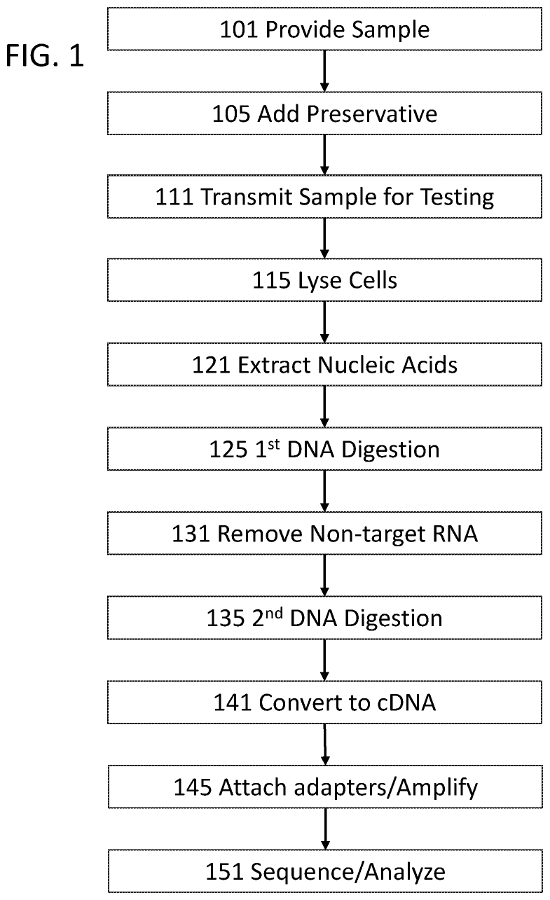

In one embodiment the method includes one or more of the following steps (referring to FIG. 1):
101 Collecting a small amount of sample, e.g., blood, e.g., from a finger prick, which can be performed using an at-home kit. The sample can be collected into, e.g., a capillary tube;
105 Adding a preservative to the sample that inhibits degradation of RNA, in particular, a preservative that will preserve the RNA at room temperature for one or more weeks;
111 Transmitting the preserved sample to a collection facility for processing;
115 Lysing cells to release polynucleotides (if necessary)
121 Extracting total nucleic acids from the preserved sample;
125 Reducing amounts of DNA from the extracted nucleic acids;
131 Removing non-target RNAs from the preserved blood sample, e.g., using DNA capture probes that capture non-informative RNAs for subsequent separation from transcriptome RNAs, to produce a transcriptome-enriched sample (this includes, for example, human genes (coding and non-coding) and any other microorganisms present in the blood sample);
135 Optionally, reducing amounts of contaminating DNA capture probes from the transcriptome-enriched sample;
141/145 Converting the RNAs in the transcriptome-enriched sample into a library of adapter tagged cDNA;
151 Sequencing adapter tagged cDNA the library and analyzing (e.g., quantifying the expression of all RNAs in the library).

A. Sample Collection

The source sample for a target analyte can be any sample that comprises the analyte. Where the object of the method is to sequence a blood transcriptome, a source material is, preferably, blood. This includes, without limitation, blood drawn from a capillary (e.g., from a capillary via skin prick (e.g., a finger prick or heel prick)), from a vein (e.g., via venipuncture) or from an artery. In the context of consumer kits, the sample is preferably, blood from a skin prick, due to its ease of collection. Blood can be collected, for example, into a capillary tube.

The amount of sample collected should be sufficient to provide sufficient amounts of target analyte for analysis. In the context of DNA analysis, e.g. sequencing, amounts of a bodily fluid such as blood can range from 1 μL to 20 mL. A vial of blood can typically collect between 5 mL to 10 mL of blood. A capillary, e.g. a glass capillary, can collect between about 5 μL to 300 μL of blood. Capillaries can be coated with an anti-coagulant, such as EDTA, which is suitable for nucleic acid analyses.

Alternatively, the collection container can be a test tube, vacuum tube for blood collection, a solid material that dries the analyte, e.g., through high surface area.

B. Preserving Target Analyte

Target analyte in the sample, such as nucleic acid, can be preserved for minimum prescribed time, for example, at least one day, at least three days, at least one week or at least one month. Such preservation is attractive if the collected sample is to be transported by common carrier to a central location for analysis. In such cases, the preservative preferably functions to preserve nucleic acids at room temperature. Preservation can be provided by adding an appropriate preservative to the sample. For example, to preserve RNA, an RNA preservative can be added to the sample. To preserve DNA, an appropriate DNA preservative can be added to the sample. In either case, the storage tube can be pre-filled with the preservative. After the sample, e.g. blood, is added, mixing can be achieved by shaking or flicking the tube multiple times. Alternatively, RNA can be preserved at low temperatures, such as by refrigeration or freezing, e.g., at −80°.

The container, such as a vial, bottle or capillary containing the sample can then be transmitted to a collection point. The container can be transmitted by hand delivery or by a common carrier, such as the US mail or a delivery service such as UPS. The collection point can be a central collection facility or laboratory.

C. Isolating Polynucleotides

On reception at a collection point, e.g., a laboratory, the sample can be processed.

Polynucleotides can be extracted directly from the sample, or cells in the sample can first be lysed to release their polynucleotides. In one method, lysing cells comprises bead beating (e.g., with zirconium beads). In another method, ultrasonic lysis is used. Such a step may not be necessary for isolating cell-free nucleic acids.

Nucleic acids can be isolated from the sample by any means known in the art. Polynucleotides can be isolated from a sample by contacting the sample with a solid support comprising moieties that bind nucleic acids, e.g., a silica surface. For example, the solid support can be a column comprising silica or can comprise paramagnetic silica beads. After capturing nucleic acids in a sample, the beads can be immobilized with a magnet and impurities removed. In another method, nucleic acids can be isolated using cellulose or polyethylene glycol.

If the target polynucleotide is RNA, the sample can be exposed to an agent that degrades DNA, for example, a DNase. Commercially available DNase preparations include, for example, DNase I (Sigma-Aldrich), Turbo DNA-free (ThermoFisher) or RNase-Free DNase (Qiagen). Also, a Qiagen RNeasy kit can be used to purify RNA.

In another embodiment, a sample comprising DNA and RNA can be exposed to a low pH, for example, pH below pH 5, below pH 4 or below pH 3. At such pH, DNA is more subject to degradation than RNA,

DNA can be isolated with silica, cellulose, or other types of surfaces, e.g., Ampure SPRI beads. Kits for such procedures are commercially available from, e.g., Promega (Madison, Wis.) or Qiagen (Venlo, Netherlands).

in certain embodiments the target RNA includes RNA anywhere in a blood sample. In such a case, cells in a blood sample can be lysed and all of the RNA isolated. In other embodiments target RNA can include cell free RNA. In such a case, cells will be removed from a sample, e.g. blood, for example by centrifugation and the remaining RNA collected.

D. Enriching for Target Molecules

Isolated polynucleotides can comprise both target species (the subject of analysis) and non-target species. Accordingly, methods of constructing nucleic acid libraries can further comprise the steps of producing a sample enriched for the target species, in which non-target species have been depleted.

1. Target Species

Determination of target species depends on the particular application. In the case of transcriptome analysis, target species can include microbial and/or host mRNA. In the case of microbiome analysis, target species may include bacterial rRNA used to identify microorganisms in the sample. In the case of genomic analysis, target species may include a selected set of genes of interest, e.g., genes associated with genetic diseases of predisposition to them, oncogenes, ancestry informative markers or short tandem repeat loci.

More specifically, in the case of transcriptome analysis, RNA species can be classified as informative, or target, RNA and non-informative, or non-target, RNA. A population of RNA molecules from a sample, e.g., a blood sample, contains many different RNA species. These different species can be ranked in terms of abundance, e.g., from most abundant to least abundant. Abundance levels span a wide dynamic range. This may include species present in hundreds of thousands of copies to species present in a few copies. Where target species include many of the less abundant species, it can be useful to reduce dynamic range of abundance by eliminating certain of the most common RNA species. Sequencing common species in a sample is not efficient and uses resources that could be used for sequencing information-providing species.

For example, in RNA taken from human blood, RNA species can be ranked from most common to least common. The quantities of each species are unevenly distributed so, when placed in rank order from most common to least common the most common species account for a much greater percentage of the total abundance of RNA in the least common species. Accordingly, when reference is made to the 90% most abundant species this refers to those species which in rank order from most common to least common account for 90% of the total abundance of the population.

Based on descending rank, about 28 of the most abundant different species account for about 95% of mRNA molecules. It is estimated that at least about 90% of RNA species in a blood sample is rRNA or tRNA. This includes, for example, microbial and/or host tRNA and rRNA. Ribosomal RNA includes, for eukaryotes, 18S and 28S rRNA and, for microbes, 16S and 23S rRNA. In the remaining mRNA, about 20 of the most abundant mRNA species account for about 90% percent of all mRNA in the blood. Among these species, mRNA encoding hemoglobin and myoglobin are the most abundant. Other common species include transcripts from genes HFM1, PDE3A, HBB, MALAT1, ATP8/ATP6, ND4L and COX1. Accordingly, in some embodiments the biological sample enriched so that human mRNA or microbial mRNA accounts for a majority of the RNA species in the sample, e.g., at least 50%, at least 60%, at least 70%, at least 80%, at least 90%, at least 95%, at least 90% or at least 99%. Human blood can contain between about 3000 and about 5000 different mRNA species corresponding to different genes that are expressed. The dynamic range of the species in a blood sample can be five orders of magnitude. This is to say; the most abundant species can be present at 100,000 times the amounts of the least abundant species. Accordingly, methods described herein enrich more rare species by about 20- to 30-fold. So, for example, this can involve removing the most abundant species in decreasing rank order (most to least abundant) that account for at least any of 90%, 91%, 92%, 93%, 94%, 95%, 96%, 97%, 98% A or to 99% of the population of RNA molecules.

2. Target Isolation

Ensembles of capture probes can be used to deplete a sample of non-target RNA, thereby enriching the sample for informative, target RNA species.

Enrichment of a sample for target species can involve positive or negative selection. In positive selection, target molecules are captured and isolated from the sample, leaving non-captured molecules. In negative selection, non-target molecules are captured and removed, leaving a sample enriched for target molecules.

A sample comprising target and non-target molecules can be enriched for target molecules by subtractive purification or enrichment. In such a process, the sample is contacted with capture probes that capture the non-target molecules. Typically, the capture probes comprise an extraction moiety which can be used as a handle to remove them with the captured non-target molecules from the sample. This approach can be used with any sort of molecular population to be partitioned. This includes, for example, populations of mixed polynucleotides, RNA populations and polypeptide populations. In the case of polypeptides capture probes can comprise antibodies attached to capture moieties.

Probes can be oligonucleotides, e.g., between about 30 and 200 nucleotides in length. Probe sequences can be selected to tile across the sequence to be captured, either in overlapping or non-overlapping format. Nucleotide sequences of tRNA and rRNA, hemoglobin, myoglobin, and other molecules to be removed from the sample, useful for developing probes to hybridize to these sequences can be found, for example at the NCBI website.

In certain embodiments, a sample comprising RNA molecules is enriched for target species by negative selection. This can comprise contacting the sample with polynucleotide probes comprising nucleotide sequences complementary to, or at least able to hybridize with, RNA or cDNA molecules having non-target sequences, capturing the non-target molecules, and using the extraction moiety to extract non-target molecules from the sample. A population of mixed species of RNA molecules can be contacted with an ensemble of poly-tagged capture probes.

E. Removing Remaining Probes

After removing labeled captures from a sample, the enriched sample may still contain unlabeled DNA probe molecules. In the case of nucleic acids, unlabeled probe molecules may interfere with subsequent analysis if they are incorporated into the library. Accordingly, methods herein provide for further reducing amounts of probe molecules in the sample. Removing remaining DNA probes can involve contacting the enriched sample with an agent that degrades DNA but not RNA. This includes, for example DNase preparations, as described herein.

After degrading the remaining DNA, the enriched RNA in the sample can be prepared into a library.

F. Preparation of Adapter Tagged Libraries

Preparing a library from RNA molecules typically comprises converting RNA into cDNA and attaching adapters.

According to one method, RNA molecules are reverse transcribed into cDNA using a reverse transcriptase. In certain embodiments, primers comprising a degenerate hexamer at their 3′ end hybridize to RNA molecules. The reverse transcriptase extends the primer and can leave a terminal poly-G overhang. In certain embodiments, the primer can also comprise adapter sequences. A template molecule comprising a Poly-C overhang and, optionally, adapter sequences, can be hybridized to the poly-G overhang and used to guide extension to produce an adapter tagged cDNA molecule comprising a cDNA insert flanked by adapter sequences.

Adapter tagged cDNA molecules can be amplified using well-known techniques such as PCR, to produce a library.

G. Sequencing and Analysis

Sequencing can proceed using any known sequencing method. High throughput sequencing methods are currently preferred. Sequencing produces sequencing reads.

Sequencing nucleic acid libraries produces sequence reads of the polynucleotides sequenced. Because nucleic acids in each nucleic acid library bear a sample barcode sequence reads can be sorted into bins based on the original library from which they are sourced. Sequence reads from individual libraries can be subject to further analysis. In one embodiment, redundant sequences can be collapsed into an original sequence, e.g., a nucleotide by nucleotide. Raw sequence reads or collapsed reads may be referred to herein as “sequenced nucleic acids”. Sequenced nucleic acids in any library can be analyzed to determine quantities of target sequences in the sample. For example, if the library comprises sequences of a microbiome, sequenced nucleic acids can be analyzed to determine species present in the sample and amount of each species.

There are many bioinformatics methods that convert raw sequences into secondary data. For example: Taxonomy classification uses databases with unique sequences belonging to different organisms. Once a sequence is matched to the database, the presence of a specific organism can be detected. By counting the sequences used to identify each organism, their relative abundances can also be measured. Functional assignments can also be made from the sequence reads. A database that correlates sequences to functions is used to convert sequencing reads into biochemical functions.

### III. Poly-Tagged Probe Ensembles

Labeled or tagged probe ensembles comprise a polynucleotide coupled to one or more labels, such as an extraction moiety or a detectable moiety. Commercial methods of synthesizing labeled polynucleotides typically are not 100% efficient. Therefore, in any ensemble of capture probes, a minority of the members do not bear an extraction moiety. For example, it is not unusual for 8%-9% of polynucleotides in a probe ensemble and obtained from commercial sources to bear no tag. In this case, such polynucleotides, and the molecules they have captured, cannot be removed from the enriched sample by typical extraction methods. They can remain, as contaminants, in the sample enriched for target molecules (“remaining probes”). Remaining probes may interfere with results by implying the presence of sequences that are not supposed to be represented in the enriched sample.

Provided herein are two methods of reducing contaminating remaining probes from a sample. These methods can be used independently or in combination. A first method involves coupling a plurality of labels to the polynucleotides so that fewer of the oligonucleotides bear no moiety. This includes coupling two, three, four or more labels to a polynucleotide molecule. A second method involves performing a subsequent reduction step that reduces the number of remaining probes in the enriched sample. This can involve, for example, degrading the DNA, e.g., using a DNase enzyme.

Accordingly, this disclosure provides poly-tagged probe ensembles. In such ensembles, substantially all probes bear at least one label, and a majority of the polynucleotides bear a plurality of labels, e.g., two, three, four or more labels. Poly-tagged probe ensembles can be synthesized by incorporating tagged nucleotides during each of a plurality of coupling steps during oligonucleotide synthesis.

Oligonucleotide probes are typically synthesized using phosphoramidite chemistry, e.g., by solid phase synthesis. In these methods, nucleotides are sequentially attached to the 5′ end of a growing chain. (Synthesis proceeds 3′ to 5′, in contrast to enzymatic synthesis, which proceeds 5′ to 3′.) In one version of this method, a growing oligonucleotide chain is attached to a solid support by a linker comprising a protecting group, e.g., 4,4′-dimethoxytrityl (DMT). A first step involves deprotecting the oligonucleotide by removing DMT (“detritylation”). A second step involves coupling the free 5′-OH of the oligonucleotide with an incoming nucleoside, provided as phosphoramidite monomer. A third step involves oxidation to stabilize the coupling. A further step involves capping unreacted oligonucleotide to prevent oligos missing a base. This is performed in iterative rounds to generate the full-length oligo sequence intended. The final oligonucleotide is cleaved from the solid support.

In typical commercial processes, coupling is less than 99% efficient, so there is a high probability that many probes in an ensemble will have capped oligos that are shorter than full length. There is also a probability that a probe ensemble will be contaminated with probes having substantial sequence non-homology with the target sequence. This can occur, for example, if a probe ensemble is synthesized on a system recently used to produce a different probe ensemble.

Labels are incorporated into probes by using an ensemble of labeled nucleotides (nucleotides modified by the attachment of a label) at one or a plurality of nucleotide coupling steps. For example, a moiety such as biotin can be pre-coupled to DNA nucleotides at a 5′ (ribose) or a 6′ (thymine base) position. By incorporating such modified DNA nucleotides into DNA polynucleotide, the biotin moiety can be incorporated at any position on the probe molecule.

However, the ensemble of labeled nucleotides coupled at each step is, itself, the product of a coupling reaction between the nucleotide and the label. This attachment step is, itself, less than 100% efficient. Accordingly, in an ensemble of “labeled” nucleotides, only about 90% of the nucleotides may actually bear the label. Therefore, in an ensemble of probes synthesized using these nucleotides at one coupling step, only about 90% of the probes may bear a label.

Accordingly, this disclosure provides a method of synthesizing probe ensembles using sequential nucleotide coupling steps in which, at each of a plurality of steps, the nucleotide ensemble used in the coupling reaction contains labeled nucleotides. It is estimated that when two nucleotide coupling steps in probe synthesis employ labeled nucleotides, about 81% of probes to bear two tags, and about 97% of all probes to bear at least one tag. It is estimated that when four nucleotide coupling steps in probe synthesis employ labeled nucleotides, about 99% of all probes to bear at least one tag. In certain embodiments of this disclosure, probe synthesis comprises at least two, e.g., at least 3, at least 4, at least 5 or at least 6 independent coupling steps using labeled nucleotide ensembles. This produces probe ensembles in which substantially all of the probes bear at least one label, and a majority bear at least 2, at least 3, at least or at least 5 labels. It is expected that some probes in the ensemble will bear no labels. Probes bearing a plurality of labels are more easily removed from a sample after hybridization with non-target sequences. Accordingly, in certain embodiments, the probes are RNA probes, which, when poly-labeled, can be effectively removed from a sample with significantly less contamination. In certain embodiments, the frequency of unlabeled probes in the ensemble is less than 5%, less than 4%, less than 3%, less than 2% or less than 1%.

In certain embodiments, the probes comprise RNA in addition to or instead of DNA. RNA probes that comprise a plurality of labels can effectively be removed from the composition by methods described herein. Such probes can be prepared either chemically, or biologically, e.g., by in vitro transcription using, for example, T7 RNA polymerase transcribed from a DNA template. In such methods, uracil can bear a capture moiety such as biotin.

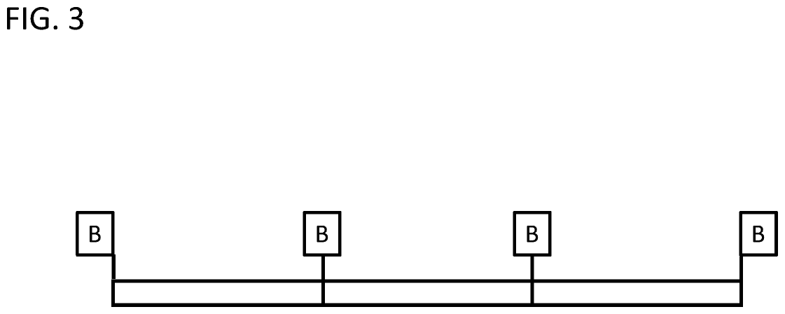

A poly-tagged probe can include at least one internal label. As used herein, an “internal label” is a label that is not attached to a terminal nucleotide of a polynucleotide probe. The label can be attached to a penultimate nucleotide in the probe. Alternatively, the label can be attached in the middle of the probe. As used herein, the “middle portion of a probe” refers to that portion more than any of 25% or 33% or 40% of the distance from either end (both ends) of the probe, or within two nucleotides of the nucleotide at the median position of the probe. Poly-tagged probes comprising internal labels can provide a further advantage of inhibiting activity of a polymerase performing primer extension on a primer bound to the probe. This, in turn, can further reduce the amount of probe sequences in in the final library. Furthermore, use of poly-tagged probe ensembles allows a method in which a second DNA removal step is not used. This may result from more probes being removed using extraction moieties and/or fewer amplified probe sequences due to blocked polymerases. Alternatively, in the case of multiple labels, labels can be spaced substantially evenly across the probe (no more than 5% deviation from even distribution). For example, three labels can be positioned substantially at the ends and at the middle of the probe. Four probes can be attached at the ends and ⅓ of the distance from either end, etc. Such an embodiment is depicted in FIG. 3. The probe can be conceptually divided into segments of substantially equal length, and labels attached at the dividing lines.

A population of mixed species of RNA molecules can be contacted with an ensemble of poly-tagged capture probes. Such an ensemble comprises a majority of probes bearing a plurality of extraction moieties. Ensembles of poly-tagged probes confer advantages over more typical probe ensembles typical probe ensembles in which a significant percentage of the probes do not bear an extraction moiety at all. Such probes cannot be extracted from the sample and, through their contamination, interfere with subsequent analysis. Poly-tagged probe ensembles include higher percentage of probes bearing at least one extraction moiety. As a result, upon extraction a higher percentage of the probes are removed from the sample.

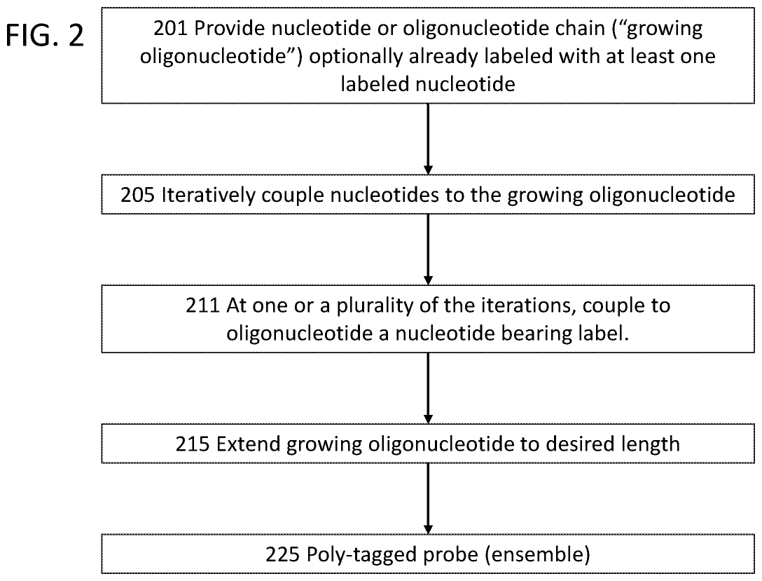

Referring to FIG. 2, a base nucleotide or oligonucleotide (collectively, in this example, “growing oligonucleotide”) is provided. In some embodiments, one or more nucleotides in the growing oligonucleotide, e.g., the base nucleotide, comprise a label (201). In certain embodiments, the growing oligonucleotide is provided attached to a solid support. To the growing oligonucleotide, nucleotides are iteratively coupled to extend the chain (205). This can be done, for example, with phosphoramidite chemistry. Unlabeled nucleotides typically are added to extend the chain. If the base nucleotide is labeled, then, in at least one coupling step, another labeled nucleotide is added to the growing oligonucleotide. If the base nucleotide is not labeled, then, labeled nucleotides are coupled at a plurality of nucleotide coupling steps (211). As the chain is extended, more unlabeled nucleotides and/or labeled nucleotides can be iteratively attached, at positions determined by the practitioner (215). In this way the final, full-length probe, bears a plurality of labels (a poly-tagged probe) (225). It is understood that, due to inefficiencies in chemistry, the final ensemble is likely to be a collection of full-length and shortened, capped probes, as well as probes bearing no, one, or a plurality of labels. The probes can be tagged with any label, including an extraction moiety. In this ensemble, a majority of the probes bear at least one label and a minority bear no labels.

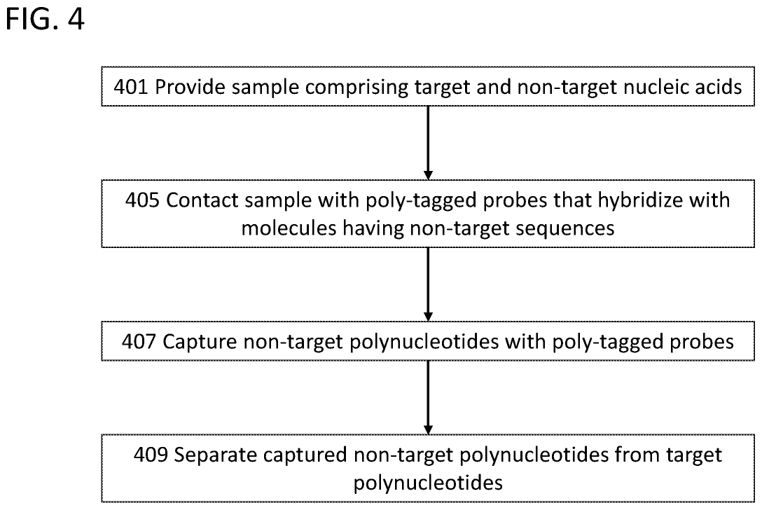

Referring to FIG. 4, a poly-tagged probe ensemble is used to deplete a sample on polynucleotides having non-target sequences. This includes providing a sample comprising nucleic acids (401). The sample is contacted with poly-tagged probes that hybridize with molecules having non-target sequences (405). Non-target sequences are captured by the probes, e.g., through hybridization (407). The captured molecules can be separated from non-captured molecules, e.g., having target sequences of interest (409).

### IV. Kits

Provided herein are kits for collection and transport of biological samples. Kits can include containers suitable to contain any biological sample as described herein. For example, liquids such as blood can be collected in a capillary or a tube. Saliva can be collected in a spit tube. Solid materials, such as skin scrapings, can be collected in a tube (e.g., a stoppered tube), a bottle or a bag. Urine can be collected in a tube and refrigerated. In certain embodiments, the samples are blood samples provided by an individual, such as a customer or consumer. Samples can be transmitted in such containers, e.g., further contained in a shipping container, to a collection facility. Kits can comprise items for sample collection from an individual, such as a lancet, scraper, a swab and a capillary tube. Containers can include compositions that inhibit degradation of RNA and/or DNA. Kits also can contain a container for shipping collected blood to a central facility, such as a box or a bag.

### EXAMPLES

### Example 1

Blood samples are collected using a lancet and an optional microcapillary tube. The sample is placed inside a tube that contains a preservative for ambient temperature transportation. In the laboratory, nucleic acids are extracted from the sample using a silica- or cellulose-based surfaces. DNA is degraded using DNase enzyme to enrich for RNA molecules. Informative RNA molecules are enriched in the sample by physically removing non-informative molecules using biotinylated DNA probes. The probes, in the case of human blood, hybridize to the most abundant transcripts, such as 45S RNA, hemoglobin, myoglobin, etc. The remaining DNA probes can, optionally, be further removed using DNase.

### Example 2

Urine is collected inside a tube, usually 50 mL conical tube, and stored in a refrigerator until it is processed. For best results, the sample should be processed within 24 hours. In the laboratory, the tube is centrifuged at 1000-5000 rpm for 15 min to pellet the microorganisms. After removing the supernatant, the pellet is resuspended in a lysis buffer (e.g. TRIzol). The rest of the process is identical to the blood process, except the DNA probes target bacterial 16S RNA and 23S RNA transcripts.

### Example 3

Respiratory samples are collected using swabs. In general, throat swabs consist of stiff handles, while nasopharyngeal swabs have longer and flexible handles. It is desirable to collect both swabs and combine them into one solution, usually containing a preservative. In the laboratory, the sample is vortexed, the swabs removed, and the solution is used to extract nucleic acids. The rest of the sample preparation is the same, except that the DNA probes target both human (e.g., 45S RNA) and bacterial (e.g. 16S RNA and 23S RNA) transcripts.

Skin samples are collected using pre-wetted swabs. A swab is rolled and swiped across a patch of skin, then placed inside a tube with a preservative. In the laboratory, the sample is vortexed, the swab is removed, and the rest of the process is identical to the sample preparation of urine.

While embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A method of preparing a cDNA library comprising:
(a) providing a sample containing RNA;
(b) optionally, disrupting cells in the sample;
(c) isolating polynucleotides from the sample;
(d) degrading initial DNA in the isolated polynucleotides to produce an RNA-enriched sample;
(e) contacting the RNA-enriched sample with an ensemble of oligonucleotide probes, wherein the oligonucleotide probes hybridize with and capture non-target RNA species in the sample and wherein the ensemble comprises oligonucleotide probes bearing two or more extraction moieties;
(f) removing captured non-target RNA species using the extraction moiety, thereby producing a target RNA-enriched sample;
(g) optionally, degrading remaining DNA in the target RNA-enriched sample;
(h) converting RNA in the target RNA-enriched sample into cDNA molecules; and
(i) attaching adapters to the cDNA molecules to produce adapter-tagged cDNA molecules, thereby producing cDNA library.

2. The method of claim 1, wherein the sample comprises RNA from a subject (e.g., human or animal).

3. The method of claim 2, wherein the subject is a human or nonhuman mammal.

4. The method of claim 2, wherein the subject is a host, and the sample comprises both host RNA and microbial RNA.

5. The method of claim 1, wherein the sample comprises a cultured biological material, an environmental sample, an agricultural sample or a forensic sample.

6. The method of claim 1, wherein the sample comprises capillary blood, venous blood or arterial blood.

7. The method of claim 1, wherein the sample comprises from about 1 μL to about 100 μL (e.g., about 5 μL to about 75 μL or about 20 μL to about 50 μL) of blood.

8. The method of claim 1, wherein the sample further comprises an RNA preservative.

9. The method of claim 8, wherein the RNA preservative comprises formalin, sulfate (e.g., ammonium sulfate) or isothiocyanate (e.g., guanidinium isothiocyanate).

10. The method of claim 1, wherein providing the sample comprises performing a skin prick and collecting the blood into a capillary tube.

11. The method of claim 10, further comprising sending the capillary tube via a common carrier to a central collection location.

12. The method of claim 1, comprising disrupting cells, e.g., by performing bead beating (e.g., with zirconium beads) or ultrasonic lysis.

13. The method of claim 1, comprising degrading initial DNA and/or remaining DNA, e.g., by treatment with a DNase (e.g., DNase I (Sigma-Aldrich), Turbo DNA-free (ThermoFisher) or RNase-Free DNase (Qiagen)).

14. The method of claim 1, wherein isolating polynucleotides comprises contacting the sample with magnetic particles (e.g., silica beads) that have nucleic acid binding affinity for bind the polynucleotides, and separating bound polynucleotides from unbound material.

15. The method of claim 1, wherein at least 90% (e.g., at least 95%, at least 96%, at least 97%, at least 98%, or at least 99%) of the oligonucleotide probes in the ensemble bear at least one extraction moiety.

16. The method of claim 1, wherein at least 50%, at least 60% or at least 75% of the oligonucleotide probes in the ensemble bear more than one extraction moiety.

17. The method of claim 1, wherein the extraction moiety is selected from the group consisting of biotin, streptavidin, avidin, a magnetically attractable particle, a peptide, and an antibody.

18. The method of claim 1, wherein non-target RNA species include one or more of: human ribosomal RNA (rRNA), human transfer RNA (tRNA), microbial rRNA, and microbial tRNA.

19. The method of claim 18, wherein non-target RNA species further include one or more of the most abundant mRNA species in the sample.

20. The method of claim 19, wherein the most abundant mRNA species removed comprise hemoglobin and/or myoglobin.

21. The method of claim 19, wherein the most abundant mRNA species removed comprise one or more of (e.g., at least 3 of, at least 4 of, at least 5 of, at least 6 of, or all of) HFM1, PDE3A, HBB, MALAT1, ATP8/ATP6, ND4L and COX1.

22. The method of claim 1, wherein captured polynucleotides represent at least 90% of polynucleotide molecules in the RNA-enriched sample.

23. The method of claim 1, wherein adapters comprise sample barcode sequences so that each adapter-tagged cDNA molecule comprises a sample barcode.

24. The method of claim 1, wherein adapters comprise sequencing platform-specific sequences necessary and/or sufficient for sequencing on a sequencing platform.

25. The method of claim 24, wherein sequencing platform-specific sequences comprise one or more of a sequencing primer hybridization site and a cluster primer binding site.

26. The method of claim 1, wherein attaching adapters comprises performing primer extension on RNA molecules using primers comprising adapter sequences or ligating adapters to double stranded cDNA molecules.

27. The method of claim 1, further comprising:
(j) sequencing the cDNA library.

28. The method of claim 27, comprising sequencing the cDNA library to a re-depth of at least 10 million reads per sample.

29. The method of claim 27, comprising pooling a plurality of different cDNA libraries, each library comprising a different sample barcode and sequencing the pooled cDNA libraries simultaneously.

30. A cDNA library comprising adaptor-tagged DNA molecules, wherein the DNA molecules comprise nucleotide sequences of RNA molecules from animal, e.g., mammalian, e.g., human, blood, and
wherein fewer than any of 50%, 40%, 30%, 20%, 10%, 5%, 4%, 2% or 1% of the sequences in the library are represented by one or more (e.g., at least three, at least four, or all of) nucleotide sequences of RNA selected from the group consisting of host rRNA, microbial rRNA, host tRNA, microbial tRNA and one or more most abundant host mRNA species.

31. The cDNA library of claim 30, further comprising trace amounts (e.g., detectable but less than 1%) of DNA probes, each probe comprising one or a plurality of extraction moieties.

32. A method of preparing a cDNA library comprising:
a) providing a sample containing DNA and RNA;
b) degrading DNA in the sample to produce an RNA-enriched sample;
c) contacting the RNA-enriched sample with oligonucleotide probes, wherein the oligonucleotide probes hybridize with and capture non-target RNA species in the sample;
d) removing captured RNA species to produce a target RNA-enriched sample;
e) degrading DNA remaining in the target RNA-enriched sample;
f) converting RNA in the target RNA-enriched sample into cDNA molecules; and
g) attaching adapters to the cDNA molecules, thereby producing a cDNA library.

33. A method of negative selection comprising:
(a) contacting a sample with an ensemble of capture probes, wherein:
(i) the capture probes selectively bind non-target molecules in the sample compared with target molecules in the sample; and
(ii) a majority of the capture probes in the ensemble bear a plurality of extraction moieties and a minority of the capture probes in the ensemble bear one or no extraction moieties; and
(b) separating bound non-target molecules from unbound target molecules by extracting capture probes with bound non-target molecules using the extraction moiety, to produce a target-enriched sample.

34. The method of claim 33, wherein a plurality of the capture probes comprise at least three, at least four or at least five extraction moieties.

35. The method of claim 34, wherein one or a plurality of the labels is an internal label not attached to a terminal nucleotide of the polynucleotide.

36. The method of claim 35, wherein the probes comprise oligonucleotide probes and the internal label is attached within the central 50%, central 40%, central 20% of the polynucleotide, or within two nucleotides of the nucleotide positioned at the median of the polynucleotide.

37. The method of claim 33, further comprising:
(c) removing un-extracted capture probes from the enriched sample.

38. The method of claim 37, wherein removing comprises degrading the un-extracted probes.

39. The method of claim 38, wherein degrading comprises degrading DNA with a DNase.

40. The method of claim 33, wherein the target molecules comprise microbial mRNA, the non-target molecules comprise RNA species selected from rRNA, tRNA and most abundant host mRNA species.

41. The method of claim 33, wherein the extraction moiety is selected from biotin, streptavidin, a magnetically attractable particle, a peptide, and an antibody.

42. An ensemble of polynucleotide probes wherein at least 90% (e.g., at least 95%, at least 96%, at least 97%, at least 98%, or at least 99%) of the probes bear at least one extraction moiety.

43. The ensemble of claim 42, wherein a majority of the probes bear at least two extraction moieties and a minority of the probes bear fewer than two extraction moieties (e.g., fewer than 50%, 40%, 30%, 20%, 10%, or 5% bear one extraction moiety and/or fewer than any of 6%, 5%, 4%, 3%, 2%, or 1% bear no extraction moiety).

44. The ensemble of claim 42 or 43, wherein at least 50%, at least 60%, at least 75%, at least 80%, at least 90%, or at least 95% of the probes in the ensemble bear at least two extraction moieties.

45. The ensemble of claim 42, wherein the polynucleotide probes comprise sequences that hybridize and bind to non-target RNA sequences.

46. A polynucleotide probe comprising a polynucleotide and a plurality of labels attached thereto, wherein one or a plurality of the labels is an internal label not attached to a terminal nucleotide of the polynucleotide.

47. The probe of claim 46, wherein the internal label is attached within the central 50%, central 40%, central 20% of the polynucleotide, or within two nucleotides of the nucleotide positioned at the median of the polynucleotide.

48. The probe of claim 46, wherein the labels are distributed substantially evenly across the probe.

49. A method of generating a poly-tagged probe comprising:
(a) providing an initial nucleotide or an oligonucleotide chain (collectively, “growing oligonucleotide”), wherein the growing oligonucleotide optionally comprises at least one nucleotide comprising a label;
(b) iteratively coupling to the growing oligonucleotide a nucleotide, wherein at one or a plurality of coupling iterations, the nucleotide coupled comprises a label, wherein a poly-tagged probe is produced.

50. The method of claim 49, wherein the nucleotide is a deoxyribonucleotide or a ribonucleotide.

51. The method of claim 49, comprising at least 3, at least 4, at least 5, at least 6 coupling steps comprising a labeled nucleotide.

52. The method of claim 49, wherein the label comprises an extraction moiety, e.g., biotin.

53. The method of claim 49, wherein the poly-tagged probes comprises at least 3, at least 4 or at least 5 labels.

54. The method of claim 49, wherein labeled nucleotides are coupled substantially evenly across the probe.

55. The method of claim 49, wherein the labeled nucleotides are coupled in a middle portion of the probe.

56. The method of claim 49, performed on an ensemble of growing oligonucleotides, wherein the ensemble comprises at least 100, at least 1000, at least 10,000, at least 100,000, or at least 1 million growing oligonucleotides.

57. The method of claim 56, wherein, after a plurality of iterative couplings (e.g., after assembly of the probes is complete) the ensemble comprises a plurality of oligonucleotides each of which comprises a plurality of labels, and a plurality of oligonucleotides, each of which comprises no more than one label, and wherein a majority of the oligonucleotides (e.g., at least 50% at least 60% at least 70% at least 80% at least 90% at least 95%) comprise a plurality of labels and a minority of the oligonucleotides (e.g., fewer than 50%, fewer than 40%, fewer than 30%, fewer than 20% fewer than 10% or fewer than 5%) of the oligonucleotides comprise no more than one label.

58. A method comprising:
(a) providing a sample comprising nucleic acid;
(b) contacting the sample with an ensemble of poly-tagged oligonucleotide probes; wherein the probes capture non-target nucleic acid molecule species in the sample;
(c) separating captured non-target nucleic acid species from target nucleic acid species.

59. The method of claim 58, wherein the probes comprise RNA oligonucleotides.

60. A kit comprising:
a) a lancet;
b) a container containing an RNA preservative; and
c) a mailing container.

61. The kit of claim 58, further comprising:
b) an EDTA-coated capillary tube.

62. The kit of claim 58, wherein the capillary tube comprises a Minivette™ point-of-care tool.

63. The kit of claim 58, wherein the kit further comprises disinfectant wipes.

64. A method comprising:
(a) providing a sample comprising polynucleotides (RNA molecules or cDNA molecules) wherein the most common polynucleotide species to the least common polynucleotide species span a dynamic range of at least any of 10³, 10⁴, 10⁵, 10⁶ or 10⁷;
(b) removing from the sample most common polynucleotide species accounting for at least 90% of the total abundance of polynucleotides to produce a sample comprising uncommon polynucleotide species; and
(c) sequencing the uncommon polynucleotide species.

65. The method of claim 64, wherein removing comprises removing species accounting for at least 99% of the total abundance.

66. The method of claim 64, wherein the low abundance polynucleotide species comprise sequences for between about 1000 and about 5000 different genes.

67. The method of claim 64, wherein removing the most common polynucleotide species does not comprise positively selecting uncommon polynucleotide species.

68. The method of claim 64, wherein the uncommon polynucleotide species comprise species within the lowest 10%, 5% or lowest 1% of abundance.
